(19) United States
(12) Patent Application Publication
(10) Pub. No.: US 2003/0190370 A1
(43) Pub. Date: Oct. 9, 2003

(54) Antibacterial agents, and antibacterial and deodorizing solution comprising the same

(76) Inventors: Sung Soo Kim, Daejeon (KR); In-Hwan Park, Daejeon (KR); Byung Chul Shin, Daejeon (KR); Soo Hong Lee, Daejeon (KR)

Correspondence Address: Pennie & Edmonds, 1155 Avenue of the Americas, New York, NY, 10036, US

(21) Appl. No.: 10/398,857

(22) PCT Filed: May 11, 2001
(86) PCT No.: PCT/KR01/00766

(30) Foreign Application Priority Data
Oct. 9, 2000 (KR) 2000=59345

(51) Int. Cl. A61K 33/38

(52) U.S. Cl. 424/618

(57) ABSTRACT

The present invention relates to novel antibacterial agents wherein the lone pair electrons of nitrogen atoms of amine compounds with high boiling point or water-soluble polymer with basic nitrogen at the backbone or side chain are coordinated with silver ion, and antibacterial and deodorizing solution comprising them. Since the antibacterial agents of the present invention has silver (Ag) ion coordinated with the lone pair electrons of nitrogen atom, the problems of conventional silver (Ag) compounds, i.e., their ease discoloration in general waters such as tap water and industrial water, are solved, and the inherent antibacterial activity is maintained due to the stabilization of silver ion (Ag+). Also, since they are highly soluble in water, they can be prepared in liquid form to be used for antibacterial and deodorizing purposes.

### BACKGROUND OF THE INVENTION

### FIELD OF THE INVENTION

The present invention relates to novel antibacterial agents wherein the lone pair electrons of nitrogen atoms of amine compounds with high boiling point or water-soluble polymer with basic nitrogen at the backbone or side chain are coordinated with silver ion, and antibacterial and deodorizing solution comprising them.

Bacteria and molds produce low-molecular compounds emitting offensive odor by decomposing organic compounds like food with the secretion of enzymes. Also, bacteria and molds are sources of offensive odor in clothes, shoes, basement, pets and ditches. Accordingly, there have been continuous attempts to develop antibacterial and deodorizing agents which are effective for deodorization, agricultural deodorizing agent, skin-related antibacterial agent, green algae prevention and cockroach extermination by exterminating microbes like bacteria.

General organic antibacterial agents, among currently used antibacterial agents, exterminate microbes by infiltrating into the microbial body and transforming its DNA. So, there is a possibility to generate resistant microbes. On the other hand, since silver (Ag) compounds exterminate microbes by disturbing the electron-transfer system of the cell membrane outside the microbial body, there is no concern of resistant microbes. Also, there is another advantage that many silver (Ag) compounds usable for antibacterial agents have much lower toxicity than general organic antibacterial agents.

Most silver (Ag) compounds have good antibacterial effect and lower toxicity compared with organic antibacterial agents, and does not induce the generation of resistant microbes. However, although insoluble silver (Ag) compounds like silver chloride (AgCl) and silver iodide (AgI) form uniform colloids and exist in ionic phase in water, precipitates are formed with time due to the inter-colloidal bond. So, they cannot be used in spray or solution form, and therefore the antibacterial effect becomes insufficient.

Soluble silver (Ag) compounds form insoluble salts like silver chloride To or silver sulfide by binding with chloride or sulfate ion generally present in water. These silver compounds transform to silver metal though reduction and oxidation by light, and then blackens to silver oxide. If soluble silver (Ag) compounds are dissolved in pure water, the discoloration is delayed. But, color changes after long-time exposure to light. Accordingly, these soluble silver (Ag) compounds are not suitable for use as everyday antibacterial agents.

For the report of everyday antibacterial agent using silver (Ag) compounds up to now, there is an example of forming silver dichloride (AgCl₂) by adding silver salt in chloride salts such as ammonium chloride, alkali metal chloride or alkaline earth metal chloride solution [International Patent Publication WO 99/09833]. In this method, silver monochloride which discolors in light is not formed when the concentration of chloride salts is at least 60 times larger than that of silver salt. Accordingly, it is not suitable for spray-type antibacterial agent since it can induce discomfort or skin irritation due to the too high salt concentration.

While silver compounds cannot be used for oral antibiotics because they are not absorbed well into the body, they can be used for antibacterial agents not required to be absorbed into the body, e.g. for the treatment of skin diseases and burns caused by bacteria and molds, since they have a wide antibacterial spectrum. However, general soluble silver compounds have problems of discoloration through the binding with skin protein, and insoluble silver compounds are not suitable for use as antibacterial agents due to their low antibacterial effect. In other words, because silver compounds are easily discolored by light, they may cause discoloration or stain when used for everyday antibacterial agents. Their colors also change when they are dissolved in water to be used for spray, skin-related antibacterial agent, agricultural fungicide, etc. The cause of discoloration is that silver ion is reduced to silver metal by light, and then this silver metal is oxidized to silver oxide by the ambient oxygen.

Also, the silver compounds can discolor skins when used as skin-related antibacterial agents.

### SUMMARY OF THE INVENTION

Generally, the reduction-oxidation reactions of soluble silver compounds in pure water without anions like distilled water proceed slowly, and therefore the discoloration time is rather long. However, general industrial water and tap water contain anions like chloride ion or sulfate ion, and they react with silver compounds to produce insoluble silver chloride or silver sulfide. These insoluble silver compounds reduce to silver metal due to the light energy of UV etc., and then oxidize by ambient oxygen to produce silver oxide and discolor the solution. So, they are not suitable for use as antibacterial deodorizing agent, agricultural antibacterial agent, green algae inhibitor, etc. Also, the soluble silver compounds may discolor the skin by binding with the skin protein and producing silver oxide when they are used for skin-related treatments.

The inventors made efforts to resolve the problems of discoloration due to light and skin discoloration while utilizing the unique antibacterial effect, low toxicity and nonresistance of silver compounds or silver ions. In doing so, aiming at the properties of silver (Ag) compounds and silver ions, we prepared silver complex with novel structure. This novel compounds has superior antibacterial effect, low toxicity, good solubility to water and stable silver ion. So, since it neither discolors by light nor discolors the skin, it can be prepared as antibacterial deodorizing solution suitable for antibacterial agents for clothes or skin treatment.

Accordingly, an object of the present invention is to provide novel antibacterial agents and antibacterial and deodorizing solution comprising them, which have superior antibacterial effect and low toxicity, and hardly discolor by light.

### BRIEF DESCRIPTION OF FIGURES

FIG. 1 is a photograph which shows the discoloration of the silver nitrate solution and silver-polyethyleneimine complex solution after exposing them for 2hr in UV.

FIG. 2 is a photograph which shows the behavior of cockroaches in the part where 2%-diluted solution of silver-polyethyleneimine complex was sprayed and in the non-treated part.

### DETAILED DESCRIPTION OF THE INVENTION

The present invention is characterized by a silver complex antibacterial agent wherein the lone pair electrons of nitrogen atoms of amine compounds with high boiling point or water-soluble polymer with basic nitrogen at the backbone or side chain are coordinated with silver ion.

Another characteristic of the present invention is an antibacterial and deodorizing solution comprising the said silver complex antibacterial agent.

Hereunder is given the more detailed description of the present invention.

The present invention relates to: a silver complex antibacterial agent which stabilizes silver ion to prevent its reaction with anions like chloride ion or sulfate ion present in water and prevents the reduction-oxidation reaction, and therefore does not discolor in general water other than distilled water; and antibacterial and deodorizing solution comprising it.

The following Formula (1) represents a silver complex wherein the polyethylenemine polymer is coordinated with silver ion, and it is a typical example of many antibacterial agents included in the present invention.

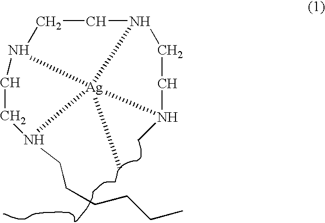

Generally, if ammonia (NH₃) is added in aqueous solution containing soluble silver compound, the discoloration is prevented. This is because the silver ion binds with the lone pair electrons of basic nitrogen to form a complex, that is suitable in ionic form itself and dissolves silver chloride which is a photo-discoloring hardly-soluble silver compound. However, ammonia (NH₃) is not suitable for use as spray-type antibacterial agent, because it emits very offensive odor. In comparison, the silver complex according to the present invention provides the silver ion stabilization effect as in the addition of ammonia (NH₃) and does not emit the offensive odor. So, it is useful for spray type antibacterial and deodorizing agent.

In the present invention, for the basic nitrogen containing material, amine compounds with high boiling point or water-soluble polymers with plenty of basic nitrogen atoms having lone pair electrons at the polymer backbone or side chain are used.

The amine compounds with high boiling point are adequate for the purpose of the present invention since they emit hardly any offensive odor. For the amine compounds with high boiling point of the present invention, any amine compounds with high boiling point including monoethanolamine, diethanolamine and triethanolamine can be used. And, they are not limited to the said examples.

For the water-soluble polymers with basic nitrogen atoms having lone pair electrons at the polymer backbone or side chain, any water-soluble polymers including polyvinylamine, polyarylamine, polyethylenemine, polyhexamethylenebiguanidine and polyvinylpyridine. And, they are not limited to the said examples.

However, polyethylene glycol, polyvinylalchol, polyacrylamide, polyviniylpyrrolidonte and polyvinylacrylic acid did not prevent the formation of silver chloride (AgCl), nor did not prevent the photo-discoloration of the solution. Accordingly, amine or amine compounds with basic nitrogen atoms having lone pair electrons are recommended for the strong coordination with the silver ion.

The water-soluble polymer with basic nitrogen atom having lone pair electrons at the backbone or side chain of the present invention can be prepared by mixing vinylamine, arylamine, ethyleneimine or vinylpyridine in organic solvents like benzene and toluene in the range of 5-20%, and radical-polymerizing them at 60-80° C. for 12-24 hours using BPO as an initiator.

For the silver (Ag) compound coordinating with basic nitrogen, the water-soluble silver compound, especially soluble in ammonia water, is recommended since it easily coordinates with polymers containing amine group. The silver compound with low toxicity is more recommendable for the purpose of the present invention. For example, silver nitrate, silver acetate, silver sulfate, silver benzoate, silver salicylate, silver thiosalicylate or silver sulfadiazine are such silver compounds. A content of silver compounds in the antibacterial agent according to the present invention is in the range of 5-50wt % of the water-soluble polymers with basic nitrogen atoms having lone pair electrons at the polymer backbone or side chain. This content can be adjusted by adjusting the number of lone pair electrons of the basic nitrogen containing amine compounds or polymers.

The present invention includes an antibacterial and deodorizing solution comprising the said silver complex as effective component. Since the antibacterial agent according to the present invention is highly soluble in water, water or mixture of water and alcohol can be used for the solvent, and the solvent can be selected depending on the purpose of use. The preparation method of the antibacterial and deodorizing solution or the effective content of the antibacterial agent is not limited specifically. Also, the common additives such as sodium benzoate, sodium salicylate or colloidal silica can be included And other additives can be easily used with the common knowledge in the art.

Because the silver complex according to the present invention easily forms complex with offensive-smelling compound or pollutant like dioxin, and prevents its vaporization. So, it helps to prevent offensive odor and environmental pollution. Especially, because silver (Ag) compounds easily forms complex with amine and thiol compounds, they can easily remove amine and thiol offensive-smelling compounds. Accordingly, since the antibacterial and deodorizing solution containing these silver complexes as effective component have superior antibacterial effect, they are useful for the removal of offensive odor caused by microbes like bacteria and molds. Also, they help to prevent the environmental pollution since they prevent the vaporization of offensive-smelling compounds and environmental pollutants by forming complex.

In addition, the silver antibacterial and deodorizing solution of the present invention is useful for the treatment of burns, prevention and treatment of bedsore and treatment of dermatitis because it provides no resistance and very low toxicity. Namely, while the general silver complexes are not suitable for the use as skin-related treatment in spite of their antibacterial effect because they react with the skin protein to discolor the skin black, the antibacterial agent of the present invention is suitable for the use as skin-related treatment in spray, ointment or gel form because it forms complex with amine polymers and the silver is kept in ionic form.

Also, the silver complex antibacterial agent of the present invention can be applied for the fungicide of vegetables and crops or green algae inhibitor of aquarium because they have superior exterminating effect of molds and algae. Generally, microbes proliferate in the steins of flowers with stems, which disturb the supply of nutrients and water through stem and therefore wilt the flowers. The antibacterial agent of the present invention extends the life of flowers by preventing the bacterial infection.

The silver complex antibacterial agent of the present invention is also useful for the extermination of insects like cockroaches. Though its cause is not certain as yet, it may be because the insects have poor digestive function compared with higher animals and can only digest low-molecular weight organic materials. Namely, if the antibacterial agent of the present invention is sprayed on food, the insects may avoid it because they cannot digest it due to the inhibition of microbial growth.

Hereunder is given the more detailed description of the present invention using examples. However, they should not be construed as limiting the scope of the present invention.

### EXAMPLE 1

### Silver-polyethyleneimine Complex

After adding 0.5 g of silver sulfadiazine, 1 g of sodium benzoate and 1 g of sodium salicylate in 1L of water, 10 g of polyethyleneimine (M.W. 25,000) was added slowly. After stirring sufficiently for 241, polyetlhyleimine complex containing silver salt was obtained.

### EXAMPLE 2

### Silver-polyhexamethylenebiguanidine Complex

After adding 5 g of polyhexametlhylenebiguanidine in 1L of water, 2 g of silver benzoate, 10 g of sodium benzoate and 1 g of sodium salicylate were slowly added while stirring. After stirring sufficiently for 24 hr, polyhexametlhylenebiguanidine complex containing silver salt was obtained.

### EXAMPLE 3

### Silver-polyvinylamine Complex

After adding 10 g of polyvinylaine and 0.5 g of colloidal silica in IL of water, 2 g of silver acetate, 1 g of silver thiosalicylate, 10 g of sodium benzoate and 1 g of sodium salicylate were slowly added while stirring. After stirring sufficiently for 24 hr, polyvinylamine complex containing silver thlosalicylate was obtained.

### EXAMPLE 4

### Silver-triethanolamine Complex

After adding 10 g of triethanolanine in 1L of water and dissolving it completely, 2 g of silver benzoate, 10 g of sodium benzoate and 1 g of sodium salicylate were slowly added while stirring. After stirring sufficiently for 24 hr, triethlanolamine complex containing silver benzoate was obtained.

### EXAMPLE 5

### Silver-diethanolamine Complex

After adding 10 g of dietlhanolamine in aqueous alcohol solution comprising 800 mL of water and 200 mL of ethanol, and dissolving it completely, 2g of silver nitrate, log of sodium benzoate and 1 g of sodium salicylate were slowly added while stirring. After stirring sufficiently for 24 hr, dietlhanolainne complex containing silver salt was obtained.

### EXPERIMENTAL EXAMPLE 1

### Investigation of Discoloration Resistance

The silver complexes obtained in the above Examples 1-5 were diluted to 2% (containing 10-40 ppm of silver ion) with tap water. Silver compounds including silver nitrate, silver sulfide, silver acetate and silver beiizoate were dissolved in tap water to 40 ppm to be used as contrast groups.

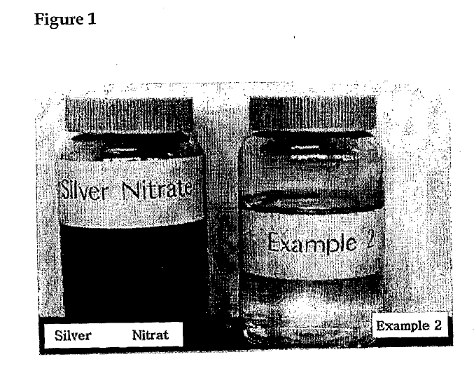

After exposing the solutions to UV for 2 hr the discoloration was investigated though eye, and the result is shown in the following Table 1. Also, the photographs after UV exposure of silver nitrate solution and silver-polyetlhyleneimine complex solution prepared in Example 2 are shown in FIG. 1.

TABLE 1

| | | Dis- |
|---|---|---|
| | Samples | coloration |
| Silver | Silver nitrate solution | 1 |
| Compounds | Silver sulfide solution | 1 |
| | Silver acetate solution | 1 |
| | Silver benzoate solution | 1 |
| Silver | Silver-polyethyleneimine complex (Example 1) | 3 |
| Complexes | Silver-polyhexamethylenebiguanidine complex | 4 |
| | (Example 2) | |
| | Silver-polyvinylamine complex (Example 3) | 3 |
| | Silver-triethanolamine complex (Example 4) | 3 |
| | Silver-diethanolamine complex (Example 5) | 3 |

Note)
1: Black precipitation formed,
2: Thick yellow,
3: Pale yellow,
4: No discoloration

As shown in Table 1 and FIG. 1, the general silver compound solution forms white colloids and then black precipitates afterwards. On the other hand, the silver complex of the present invention shows little photo-discoloration.

### EXPERIMENTAL EXAMPLE 2

### Investigation of Minimum Growth Inhibition Concentration

The minimum growth inhibition concentration was measured using test-tube dilution method in order to evaluate the antibacterial activity of the silver complexes obtained in Examples 1-5. The result is shown in the following Table 2.

TABLE 2

| | Minimum Growth Inhibition Concentration (ppm) | | | | |
|---|---|---|---|---|---|
| Microbes | Example 1 | Example 2 | Example 3 | Example 4 | Example 5 |
| E. coli (Escherichia coli | <1 | <1 | <1 | 1 | 1 |
| ATCC 25922) | | | | | |
| E. coli O-157 | 1 | 1 | 1 | 3 | 3 |
| (Escherichia coli | | | | | |
| ATCC 43895) | | | | | |
| Staphylococcus aureus | 2 | 3 | 3 | 5 | 6 |
| ATCC 25923 | | | | | |
| Staphylococcus aureus | 3 | 3 | 3 | 5 | 7 |
| ATCC 6538P | | | | | |
| Trichophyton rubrum | 1 | 2 | 2 | 2 | 2 |
| ATCC 28188 | | | | | |
| Algae (Candida Albicans | 1 | 1 | 1 | 1 | 1 |
| ATCC 11651) | | | | | |
| Yeast (Trentopholia odorata) | <1 | <1 | <1 | <1 | <1 |

As shown in Table 2, all the silver complexes according to the present invention have superior antibacterial effect.

### EXPERIMENTAL EXAMPLE 3

### Investigation of Antibacterial Effect on Fabrics

After diluting the silver complexes obtained in Examples 1-3 to 2% (containing 10-40 ppm of silver ion) with aqueous alcohol solution comprising 9:1 of tap water and ethanol, it was sprayed on cotton fabric. After drying the fabric, its antibacterial effect was investigated. The result is shown in the following Table 3.

The samples used for antibacterial effect evaluation test were 1 g respectively, and their size was 10×10×1min³. The antibacterial effect evaluation test followed the bacteria count method (KS K 0693) using E. coli ATCC 25922 and Stapltylococcits aireus ATCC 6538. The bacteria culture time was 24 hr.

TABLE 3

| | E. coli | | | Staphylococcus aureus | | |
|---|---|---|---|---|---|---|
| | Initial | Number | Microbial | | Number | |
| | Number | of | Reduction | Initial | of | Microbial |
| | of | Microbes | Ratio | Number of | Microbes | Reduction |
| Samples | Microbes | after Test | (%) | Microbes | after Test | Ratio (%) |
| Example 1 | 1.0 × 10⁷ | 0 | 100 | 1.9 × 10⁷ | 2.3 × 10⁴ | 99.9 |
| Example 2 | 1.0 × 10⁷ | 6,000 | 99.9 | 1.9 × 10⁷ | 0 | 100 |
| Example 3 | 1.0 × 10⁷ | 9,000 | 99.9 | 1.9 × 10⁷ | 3.4 × 10⁴ | 99.8 |

As shown in Table 3, all the silver complexes according to the present invention showed superior antibacterial effect on fabrics.

### Experimental Example 4

Evaluation of Fungus Resistance on Fabrics

After diluting the silver complexes obtained in Examples 1-3 :to 2% (containing 10-40 ppm of silver ion) with aqueous alcohol solution comprising 9:1 of tap water and ethanol, it was sprayed on cotton fabric. The fungus resistance of the fabric was investigated. The result is shown in Table 5.

The base sample used for fungus resistance evaluation test was 30×30×1mm³. The fungus resistance evaluation test followed the mold resistance test method (KS A 0702) using ATCC 10254 (Aspeigillus niger ATCC 10254) black mold. The bacteria culture time was 2 weeks.

The mold resistance is presented in the following Table 4.

TABLE 4

| Growth of Hypha | Mold Resistance |
|---|---|
| No hypha growth was identified in the inoculation part | 3 |
| of the sample. | |
| The hypha growth area of the inoculation part was | 2 |
| smaller than ⅓ of the entire area. | |
| The hypha growth area of the inoculation part was | 1 |
| larger than ⅓ of the entire area. | |

The result of the fungus resistance evaluation test is shown in the following Table 5.

TABLE 5

| Samples | Evaluation Result |
|---|---|
| Example 1 | 3 |
| Example 2 | 3 |
| Example 3 | 3 |

As shown in Table 5, the silver complexes according to the present invention have superior mold resistance of 3.

### EXPERIMENTAL EXAMPLE 5

### Investigation of Deodorizing Effect

After inserting 1 mL of acetic acid respectively in two sealed acryl boxes (30×30×30 cm³), they were left for 2 hr for the acetic acid to vaporize sufficiently. About 1 mL of 2% silver complex solution (containing 40 ppm of silver ion) obtained from Table 2 was sprayed in one box, and the other was used as a contrast group. After 30 min, gases were collected from each box to investigate the amount of acetic acid through gas chromatography.

With the same method, deodorizing effect was investigated about thiocresol. The result is shown in the following Table 6.

TABLE 6

| | Peak Area of Gas Chromatography (Arbitrary Unit) | |
|---|---|---|
| Samples | Acetic Acid | Thiocresol |
| Contrast Group | 340 | 420 |
| Example 2 | 20 | 35 |

As shown in Table 6, while there was a lot of vaporized acetic acid and thiocresol, the use of antibacterial and deodorizing agent of the present invention removed most of acetic acid and tlhiocresol, which are the cause of offensive odor.

### EXPERIMENTAL EXAMPLE 6

### Evaluation of Flower Wilting Prevention Effect

In two vases with the same size 300 mL of tap water was poured. 1 mL of 2% solution (containing 10 ppm of silver ion) of the silver complex obtained from Example 1 was added to one of the two vases and the other was used as a contrast group. After putting 10 as picked roses in each vase at the same place, the condition of flowers was checked.

The result showed that the flowers in the vase where the antibacterial and deodorizing agent of the present invention was added wilted later than 7 days compared with the contrast group. And, while there was offensive smell in the water of the contrast group vase, the one in the vase wherein the antibacterial and deodorizing agent was added had no offensive odor at all.

### EXPERIMENTAL EXAMPLE 7

Evaluation of Cockroach Extermination Effect

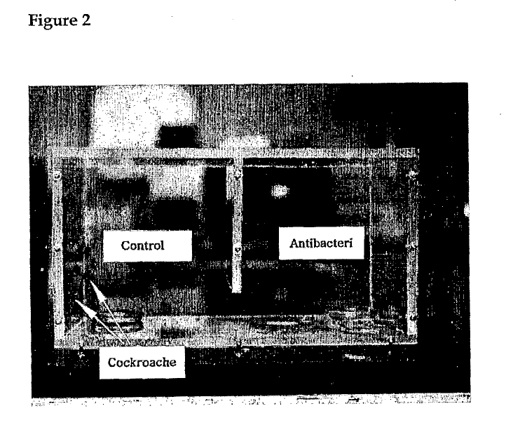

In each side of an acryl box (25×50×25cm³) comparted at the center, bread without sugar was inserted. 2% solution (containing 10 ppm of silver ion) of the silver complex obtained from Example 1 was sprayed on one side, and the other was used as a contrast group. After inserting 20 cockroaches in the box, their behavior was observed. The result showed that while all the cockroaches did not stay long at the bread stuck with the antibacterial and deodorizing agent (Example 1) of the present invention, they stayed long and eat the bread in the contrast group. Also, most cockroaches inhabited the chamber where the antibacterial and deodorizing agent was not sprayed. The result is shown in FIG. 2.

### EXPERIMENTAL EXAMPLE 8

### Preparation of Ointment

After mixing 1 wt % of silver complex obtained from Example 1, 2 wt % of wax, 63 wt % of pure water and 25 wt % of glycerin, they were heated to 65-70° C. while stirring for good mixing and dissolving. After adding 10 wt % of glyceryl monostearate and heating to 65-70° C., it was stirred with the mixture sufficiently to obtain an ointment.

The fungus resistance was investigated with the same method in Experimental Example 4 to evaluate its efficiency. As a result, it showed very superior antibacterial effect with rating 3.

As explained in detail above, while most silver compounds cannot be used for everyday antibacterial agent in spite of their good antibacterial effect because their color easily changes in common water like tap water or industrial water and discolor the skin; the antibacterial agent of the present invention is a silver complex wherein the lone pair electrons of nitrogen atom in the amine compounds with high boiling point or water-soluble polymers with basic nitrogen are coordinated with silver ion, and it has superior discoloration resistance and antibacterial effect, and highly soluble in water. So, when it is used in liquid form dissolved in water or mixture of water and alcohol, it can be used effectively for removing offensive odor, agricultural fungicide, environmental pollutant elimination, prevention and treatment of bedsore, treatment of dermatitis, antibacterial disinfection, prevention of flower wilting, extermination of cockroaches and prevention of green algae.

What is claimed is:

51. An antibacterial and deodorizing solution comprising the a silver complex, wherein the lone pair electrons of nitrogen atom of amine compound with high boiling point or water-soluble polymer having basic nitrogen atom in the backbone or side chain are coordinated with silver ion, as effective component.

62. The antibacterial and deodorizing solution according to claim 5, wherein said silver complex is dissolved in water solvent or mixture solvent of water and alcohol.

73. The antibacterial and deodorizing solution according to claim 5, wherein said water-soluble polymer having basic nitrogen atom in the backbone or side chain is selected from the group consisting of polyvinylamine, polyarylamine, polyethyleneimine, polyhexamethylenebiguanidine and polyvinylpyridine.

84. The antibacterial and deodorizing solution according to claim 5, wherein said amine compound with high boiling point is selected from the group consisting of monoethanolamine, diethanolamine and triethanolamine.

95. The antibacterial and deodorizing solution according to claim 5, wherein said silver ion is derived from silver nitrate, silver acetate, silver sulfate, silver benzoate, silver salicylate, silver thiosalicylate or silver sulfadiazine.
